(19) United States
(12) Patent Application Publication
(10) Pub. No.: US 2011/0189342 A1
(43) Pub. Date: Aug. 4, 2011

(54) HIGH-PURITY GALACTOOLIGOSACCHARIDES AND USES THEREOF

(76) Inventors: Hea-seok JEONG, Icheon-City (KR); Hyuk-kon Kwon, Icheon-City (KR); Jong-Soo Choi, Icheon-City (KR)

(21) Appl. No.: 12/714,777

(22) Filed: Mar. 1, 2010

Related U.S. Application Data
(60) Provisional application No. 61/300,317, filed on Feb. 1, 2010.

(51) Int. Cl. C12P 19/00; A23C 9/12

(52) U.S. Cl. 426/42; 435/72; 426/61

(57) ABSTRACT

Disclosed herein are high-purity galactooligosaccharide compositions, methods of producing high-purity galactooligosaccharides, food products and method of preparing food products comprising a high-purity galactooligosaccharide composition.

### CROSS-REFERENCE TO RELATED APPLICATIONS

This application claims priority to U.S. Provisional Patent Application No. 61/300,317, filed Feb. 1, 2010, the entire contents of which are hereby incorporated by reference in its entirety.

### TECHNICAL FIELD

The present technology relates generally to methods of processing food and industrial products. In particular, the present technology relates to the field of high-purity galactooligosaccharide (GOS) compositions, methods of producing them, food products comprising high-purity GOS compositions and methods of making such food products.

### BACKGROUND

The following description is provided to assist the understanding of the reader. None of the information provided or references cited is admitted to be prior art to the present technology.

Oligosaccharides are used in many food and feed formulations for their benefits for digestive health and immune enhancing properties. Oligosaccharides such as fructooligosaccharides (FOS) and galactooligosaccharides (GOS) are prebiotics that are fermented by the probiotic bacteria in the colon into short-chain fatty acids, leading to improved intestinal microflora and other health benefits such as improved mineral absorption. In addition, oligosaccharides act as soluble fibers, delivering flavor enhancement benefits, moisture retention and shelf-life extension properties. The health and functional benefits of oligosaccharides depend on product source and physicochemical structure.

The composition of the galactooligosaccharide fraction varies in chain length and type of linkage between the monomer units. The composition may be comprised of β-(1,3) galactosyl linkages, β-(1,4) and β-(1,6) linkages. The structure of the GOS produced depends primarily upon the source of enzymes amongst other things. For example, the general structure of a β-(1,4) linked galactooligosaccharide molecule is illustrated as Structure I,

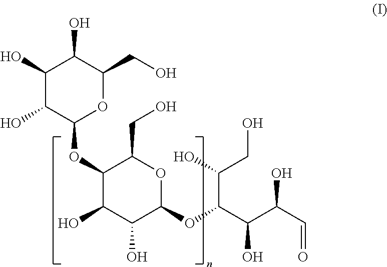

Human milk oligosaccharides (HMO) are resistant to enzymatic digestion in the stomach and small intestine, reaching the colon intact where they are fermented by colonic microflora. HMO have been found to be similar in their resistance to digestion as lactulose by hydrogen breath test in breast-fed infants. Human milk contains a complex mixture of more than 1000 different oligosaccharides mostly of low molecular weight. However, only 130 of them have been identified. Besides 7% lactose, human milk contains approximately 1% oligosaccharides. The backbone structure of these oligosaccharides is based on lactose, with added galactose units to form galactosyl-lactoses, namely 3′-galactosyl-lactose, 4′-galactosyl-lactose and 6′-galactosyl-lactose. Oligosaccharides are elongated by repeated units of galactose-N-acetylglucosamine attached to core lactose and further modified by addition of functional groups such as fucose and sialic acid.

Beneficial strains of bacteria have been shown to ferment GOS, particularly Bifidobacteria and Lactobacilli species, which are known to produce short chain fatty acids (SCFA). SCFA promote protective effects on the gut and host, such as increased gut integrity, enhanced immunity through pathogen inhibition, reduction of putrefactive substances and improved bowel function.

Since neither the major putrefactive nor pathogenic bacteria in the gut ferment GOS, it fosters an environment that favors competitive inhibition of pathogens. When Bifidobacteria species increase, pathogens tend to decrease in number. GOS increase Bifidobacteria and Lactobacilli and decreased pathogenic bacteria in vitro. Moreover, human strains of pathogenic species of Streptococcus and Campylobacter exhibit minimal to no ability to ferment GOS in vitro. In addition to substrate studies, the bifidogenic properties of GOS have been documented across several species in vivo, including humans.

GOS may inhibit infectious bacteria by several methods. One method, which is distinct from other methods employed by most oligosaccharides, is an anti-adhesive mechanism that promotes pathogen exclusion. The structure of GOS resembles the receptor sites coating the epithelial cells recognized and adhered to by intestinal pathogens. Instead of binding to a host cell surface that would initiate the infection process, the pathogen binds to the soluble decoy GOS, and then is displaced or flushed from the gastrointestinal tract. Preventing this initial adherence may ultimately inhibit the infection process. Adherence inhibition of GOS is dose dependent, so the greater the purity, the smaller the dose needed to produce efficacious results.

Calcium absorption may also be improved with GOS through the production of SCFA. In the gastrointestinal tract, minerals must be kept soluble in order to be absorbed and solubility is reduced at higher pH levels. The production of SCFA lowers luminal pH to an optimal level for keeping minerals in solution longer, thereby enhancing their absorption. Marini et al., observed similar calcium/phosphorus ratio in the urine of infants fed formula with GOS/FOS mixture as with infants fed human milk suggestive of an influence on calcium absorption. In a study with postmenopausal women, a significant increase in calcium absorption was shown with 12 g of GOS daily.

### SUMMARY

In accordance with one aspect, the present technology relates to high-purity galactooligosaccharide compositions and methods for producing them. In another aspect, the present technology relates to food products made with high purity GOS.

In one embodiment, the present disclosure provides a method of producing galactooligosaccharide comprising catalyzing the conversion of lactose to galactooligosaccharide using a suitable enzyme; and chromatographically purifying the galactooligosaccharide using an ion exchange resin comprising a K⁺ counter ion.

In one embodiment, the enzyme is a β-galactosidase. In some embodiments, the enzyme is derived from a host-cell selected from the group consisting of Bifidobacterium, Lactococcus, Lactobacillus, Streptococcus, Leuconostoc, Escherichia, Bacillus, Streptomyces, Saccharomyces, Kluyveromyces, Candida, Torula, Torulopsis and Aspergillus. In some embodiments, the β-galactosidase is derived from Cryptococcus laurentii, Aspergillus oryzae, Aspergillus nigar; Bacillus circulans, Bacillus subtilis, Bacillus licheniformis, Lactobacillus bulgaricus, Streptococcus thermophilus, Bullera singularis, Bifidobacterium breve, Bifidobacterium longum, Bifidobacterium infantis, Bifidobacterium bifidum, Lactococcus lactis, Candida pseudotropicalis, or Kluyveromyces lactis. In an illustrative embodiment, the β-galactosidase is derived from Bacillus circulans. In some such embodiments, the β-galactosidase is derived from Bacillus circulans LOB 377.

In one embodiment, the ion exchange resin comprising a K⁺ counter ion is an acidic cation exchange resin with cross-linked polystyrene matrix and sulfonate functional groups.

In some embodiments, the method further comprises a ion-exchange purification process. In some embodiments, the ion-exchange purification process is conducted after the conversion of lactose to galactooligosaccharide and before the chromatographic purification step. In some embodiments, the process comprises the step of passing the GOS solution through atleast one column selected from a cation column with strongly acidic cation exchange resin, an anion column with a basic anion exchange resin, and a mixed bed column comprising a combination of both strongly acidic and strongly basic resins. In some embodiments, the basic anion exchange resin is selected from a weakly basic anion exchange resin and an intermediate basic anion exchange resin. Thus, in some embodiments, the basic anion exchange resin is a weakly basic anion exchange resin. In other embodiments, the basic anion exchange resin an intermediate basic anion exchange resin

In some embodiments, the method comprises a three-column ion-exchange process. In some embodiments, the three-columns comprise a cation column with strongly acidic cation exchange resin, an anion column with a basic anion exchange resin, and a mixed bed column comprising a combination of both strongly acidic and strongly basic resins. In some embodiments, the basic anion exchange resin is selected from a weakly basic anion exchange resin and an intermediate basic anion exchange resin. In some embodiments, the three-column ion-exchange purification process is conducted after the conversion of lactose to galactooligosaccharide. In some such embodiments, the three-column ion-exchange process is conducted prior to the chromatographic purification step.

In some embodiments, the method further comprises the additional step of decolorizing the GOS solution with activated carbon. In some embodiments, the decolorization step is conducted prior to the three-column ion-exchange purification process.

In some embodiments, an aqueous solution of lactose is contacted with the enzyme. In some embodiments, the concentration of lactose in the solution ranges between about 1% and about 100%. In some embodiments, the concentration of lactose in the solution ranges between about 5% and about 90%. In other embodiments, the concentration of lactose in the solution ranges between about 5% and about 45%. In some embodiments, the galactooligosaccharide composition comprises no more than about 10% by weight of carbohydrates other than galactooligosaccharide.

In some embodiments, the method is included in a process for producing a baby food, an infant formula, a beverage, a yogurt, or a dietary supplement.

In one embodiment, the present disclosure provides a method for producing high-purity galactooligosaccharide, the method comprising:

(a) contacting lactose with a suitable enzyme under mildly acidic conditions to produce galactooligosaccharide;

(b) decolorizing galactooligosaccharide with activated carbon;

(c) passing galactooligosaccharide through one or more ion-exchange columns selected from a cation column with strongly acidic cation exchange resin, an anion column with a basic anion exchange resin, and a mixed bed column comprising a combination of both strongly acidic and strongly basic resins; and

(d) chromatographically purifying the galactooligosaccharide using an ion exchange resin comprising a K⁺ counter ion.

In another aspect, a galactooligosaccharide composition obtained by the present methods is provided. In some embodiments, the galactooligosaccharide composition comprises, as effective constituents, a mixture of one or more disaccharides, trisaccharides, tetrasaccharides and pentasaccharides. In illustrative embodiments, the composition comprises from about 10% to about 25% w/v of the disaccharide, from about 30% to about 50% w/v of the trisaccharide, and from about 30% to about 45% w/v of the tetrasaccharide and higher oligosaccharides.

In yet another aspect, a food product is provided comprising a galactooligosaccharide composition. In some embodiments, the food product comprises (a) a galactooligosaccharide composition comprising no more than about 10% by weight carbohydrates other than galactooligosaccharide; and (b) at least one additional edible ingredient. In some embodiments, the galactooligosaccharide composition in the food product comprises no more than about 8% by weight of carbohydrates other than galactooligosaccharide. In some embodiments, the composition further comprises one or more oligosaccharides selected from the group consisting of fructooligosaccharides, isomaltooligosaccharides, and inulin.

Generally, the present food products include a oligosaccharide composition and at least one additional edible ingredient. In some embodiments, the food products may also include a bulking agent. In some embodiments, the GOS compositions are characterized in that they include no more than about 15% by weight of carbohydrates other than GOS, no more than about 10% by weight of carbohydrates other than GOS, no more than about 8% by weight of carbohydrates other than GOS, and no more than about 5% by weight of carbohydrates other than GOS. As such, the present food products have better properties due to the lower inclusion level required to obtain an effective amount of GOS in the final food product.

The GOS product can be further modified to modify or add additional functional groups. For e.g., the composition of the GOS product obtained can be further modified enzymatically to closely mimic the human milk composition. Human milk contains about 7% lactose, which is also present in the same amount in the instant GOS product. Further, the backbone structure of the oligosaccharides in human milk is based on lactose, with added galactose units to form galactosyl-lactoses, namely 3′-galactosyl-lactose, 4′-galactosyl-lactose and 6′-galactosyl-lactose. Using suitable enzymes, oligosaccharides in the GOS product can be elongated by repeated units of galactose-N-acetylglucosamine attached to core lactose and further modified by addition of functional groups such as fucose and sialic acid.

Non-limiting examples of food products that may be made with the high purity GOS compositions include infant formula, baby and toddler foods, beverages, yogurts, and nutritional supplements. The food product may take the form of a dry powder and include at least one salt or acid.

In one aspect, a method of preparing a food product comprising a galactooligosaccharide composition is provided. In some embodiments, the method comprises mixing a galactooligosaccharide composition comprising no more than about 10% by weight of carbohydrates other than galactooligosaccharide and at least one additional edible ingredient.

In some embodiments, the food product comprising galactooligosaccharide is a beverage and the additional edible ingredient comprises at least one protein or protein hydrolysate, vegetable oil, and at least one additional carbohydrate. In some embodiments, the food product comprising galactooligosaccharide is yogurt and the additional edible ingredients comprise at least one protein or protein hydrolysate, milk, at least one acid, and at least one additional carbohydrate. In some embodiments, the food product comprising galactooligosaccharide is a baby food and the additional edible ingredients comprise at least one protein or protein hydrolysate and at least one additional carbohydrate. In some embodiments, the food product comprising galactooligosaccharide is an infant formula and the additional edible ingredients comprise at least one protein or protein hydrolysate, vegetable oil, and at least one additional carbohydrate. In some embodiments, the food product comprising galactooligosaccharide is a dietary supplement and the additional edible ingredients comprise a pharmaceutically acceptable carrier.

The foregoing summary is illustrative only and is not intended to be in any way limiting. In addition to the illustrative aspects, embodiments, and features described above, further aspects, embodiments, and features will become apparent by reference to the following detailed description.

### BRIEF DESCRIPTION OF THE FIGURES

FIG. 1 is a flow chart showing the illustrative steps for the preparation of the high purity GOS composition of the present technology.

FIG. 2 is a flow chart showing an illustrative process for the preparation of a beverage composition using the high-purity GOS composition of the present technology.

FIG. 3 is a flow chart showing an illustrative process for the preparation of a yogurt composition using the high-purity GOS composition of the present technology.

### DETAILED DESCRIPTION

In the description that follows, a number of terms are used extensively. Definitions are provided to facilitate understanding of the technology. The terms described below are more fully defined by reference to the specification as a whole. Units, prefixes, and symbols may be denoted in their accepted SI form.

The terms “a” and “an” as used herein mean “one or more” unless the singular is expressly specified.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the enumerated value.

The term “carbohydrate” is used interchangeably with the terms “saccharide,” “polysaccharide,” “oligosaccharide” and “sugar” which are further described below.

As used herein, the terms “fructooligosaccharide” or “FOS” refer to a compound comprising three or more monosaccharides, independently selected from the group consisting of glucose and fructose. In one embodiment, FOS may be expressed by a general formula Glc-(Fru)ₙ (where Glc represents a glucose residue, Fru represents a fructose residue, and n represents an integer between 1 and 8, typically between 2 and 4).

As used herein, the terms “galactooligosaccharide” or “GOS” refer to a compound comprising three or more monosaccharides, independently selected from the group consisting of glucose and galactose. In one embodiment, GOS may be expressed by a general formula Gal-(Gal)ₙ-Glc (where Gal represents a galactose residue, Glc represents a glucose residue, and n represents an integer between 1 and 10, typically between 1 and 8, and preferably between 2 and 6).

As used herein, the term “chromatographic purification” refers to any technique for the separation and purification of various fractions of GOS products. Therefore, “chromatographic purification” as used herein encompasses liquid-liquid fractionation or liquid-liquid partitioning of the GOS products by which separation, fractionation, concentration and/or purification of the GOS product can be achieved.

As used herein, the term “purity” refers to the weight percentage of a particular compound present in a composition. Thus, in some embodiments, a carbohydrate composition will comprise GOS in a particular purity, with the remainder of the composition comprising a mixture of other mono-, di-, and oligo-saccharides. In some embodiments, the purity of the GOS composition may range from about 70% to about 100%; from about 80% to about 100%; or from about 90% to about 100%. In certain embodiments, the preparation is at least about 92% pure, at least about 93% pure, at least about 94% pure, at least about 95% pure, at least about 96% pure, at least about 97% pure, at least about 98% pure, at least about 99% pure, at least about 99.5% pure, or at least about 99.9% pure on a dry weight basis. In illustrative embodiments, the product has a GOS purity of at least about 90% on a dry weight basis. Purity can be measured according to any method known to those of skill in the art, including, e.g., liquid chromatography.

As used herein, the term “saccharide” refers to a carbohydrate which is a polyhydroxy aldehyde or ketone, or derivative thereof, having the empirical formula (CH₂O)ₙ wherein n is a whole integer, typically greater than three. Monosaccharides, or simple sugars, consist of a single polyhydroxy aldehyde or ketone unit. Exemplary monosaccharides include glucose, mannose, xylose, galactose, fucose, fructose, sialic acid, N-acetyl glucosamine and N-acetyl galactose-amine. Disaccharides contain two such units joined by a glycosidic linkage. Disaccharides include, for example, sucrose, lactose, maltose and cellobiose. Oligosaccharides typically contain from three to ten monosaccharide units each joined by a glycosidic linkage. Exemplary poly- or oligo-saccharides include FOS, GOS, lactosucrose, isomaltulose, glycosyl sucrose, isomaltooligosaccharide, gentioligosaccharide, xylooligosaccharide, and combinations thereof. Trisaccharides are oligosaccharides composed of three monosaccharides with two glycosidic bonds connecting them. Trisaccharides include, for example, raffinose, melezitose and maltotriose. Trisaccharides include, for example, acarbose and stachyose. Polysaccharides (glycans) typically contain more than ten monosaccharide units. The term “sugar” generally refers to mono-, di- or oligo-saccharides.

As used herein, a “subject” or “patient” is a mammal, such as a cat, dog, rodent or primate. Typically, the subject is a human. The term “subject” and “patient” can be used interchangeably.

Disclosed herein are high-purity galactooligosaccharide compositions, methods of producing them, food products comprising a high purity oligosaccharide compositions and methods of making food products comprising a high purity oligosaccharide composition. The high-purity GOS compositions can be added to food at relatively low inclusion levels, thereby providing quality food compositions comprising an effective amount of GOS. The uniqueness of this technology relies at least in part on a high purity GOS composition and its effectiveness in regard to inclusion levels.

High-purity GOS compositions have several advantages. First, these purity levels allow for a small amount of the GOS composition be added in order to obtain an effective amount of GOS, i.e., the compositions have low inclusion rates. Low inclusion levels are often cost effective and do not require significant alterations to food and supplement formulations when they are added. Second, high purity oligosaccharide compositions contain low levels of residual sugars in the form of lactose, galactose and glucose.

Given its unique characteristics, GOS can be used to deliver digestive, immune and mineral absorption benefits to several applications, including beverages, dairy products, baby and toddler foods, infant formula and supplements. Considering the body of science that supports the health benefits for GOS, a high purity product can be added at effective inclusion levels. The GOS compositions (with a purity level from about 86% to about 100%) may be added to foods at an inclusion level, for example, from about 0.01% to about 1%, from about 0.1% to about 1%, from about 0.5% to about 1%, from about 0.5% to about 5%, or from about 1% to about 10%. The GOS compositions (with a purity level from about 86% to about 100%) may also be provided in the form of a nutritional or dietary supplement, e.g., a capsule, in substantially pure form, or further comprising a pharmaceutically acceptable carrier.

### Production of High Purity GOS

In accordance with one aspect, the present technology relates to high-purity galactooligosaccharide (GOS) compositions and methods for producing them. GOS can be produced either by enzymes or by fermentation using microorganisms. In one embodiment, GOS may be manufactured from lactose using enzymatic conversion and purification techniques.

Thus, in one embodiment, a method of producing galactooligosaccharide is provided, which may comprise catalyzing the conversion of lactose to galactooligosaccharide using a suitable enzyme; and chromatographically purifying the galactooligosaccharide using an ion exchange resin comprising a K⁺ counter ion.

The substrate, i.e., lactose, required for the process, may be obtained from various sources known in the art. In some embodiments, the lactose is manufactured from milk (e.g., bovine edible lactose) or from milk products (e.g., whey) using multiple combinations of enzymatic and purification steps. The lactose used can be food grade or pharmaceutical grade. In some embodiments, the lactose can be dissolved in a suitable solvent such as e.g. heated water. In some embodiments of the method of producing galactooligosaccharides, an aqueous solution of lactose is contacted with a suitable enzyme. In some embodiments of the method of producing galactooligosaccharides, the lactose is contacted with a solution of the enzyme.

Any suitable enzyme or microorganism known in the art, which can convert lactose to GOS, may be employed in the present methods. In some embodiments, the enzyme may be a β-galactosidase, and the microorganism may be capable of producing β-galactosidase. In some embodiments, the β-galactosidase may be obtained from a non-toxigenic, non-pathogenic microorganism. In some embodiments, the enzyme is derived from a host-cell selected from the group consisting of Bifidobacterium, Lactococcus, Lactobacillus, Streptococcus, Leuconostoc, Escherichia, Bacillus, Streptomyces, Saccharomyces, Kluyveromyces, Candida, Torula, Torulopsis and Aspergillus. In some embodiments, the β-galactosidase is derived from Cryptococcus laurentii, Aspergillus oryzae, Aspergillus nigar; Bacillus circulans, Bacillus subtilis, Bacillus licheniformis, Lactobacillus bulgaricus, Streptococcus thermophilus, Bullera singularis, Bifidobacterium breve, Bifidobacterium longum, Bifidobacterium infantis, Bifidobacterium bifidum, Lactococcus lactis, Candida pseudotropicalis, or Kluyveromyces lactis. In an illustrative embodiment, the β-galactosidase is derived from Bacillus circulans. In some such embodiments, the β-galactosidase is derived from Bacillus circulans LOB 377. β-galactosidases are generally known as enzymes that catalyze the hydrolysis of β-D-galactopyranoside such as lactose, however, the enzyme also catalyzes transgalactosylation of these sugars, and when lactose is present at high concentrations, the transgalactosylation reaction predominates.

In some embodiments, the method may be a multi-step process comprising enzymatic conversion and purification steps. In some embodiments, the substrate, i.e., lactose, is first dissolved in a suitable solvent such as, e.g., heated water, to obtain a desired concentration. In some embodiments, the concentration of said lactose in the solution may range between about 1% and about 100%. In some embodiments, the concentration of said lactose in the solution may range between about 5% and about 90%. In other embodiments, the concentration of said lactose in the solution may range between about 5% and about 45%. The temperature of the solution may be raised or reduced as desired and the pH may be adjusted to mildly acidic using suitable acids or bases. The β-galactosidase is then added to the solution where it reacts with lactose to produce GOS. The reaction period may vary from a few hours to a few days depending on the desired oligosaccharide content. After the desired oligosaccharide content is reached, the enzymes in the GOS solution are then deactivated by heating the solution at a temperature and for a time within enzyme deactivation conditions, whereby the enzyme is deactivated. The inactivated enzyme can be removed via filtration using suitable enzyme filters such as, e.g., a Celite filter (plankton diatomite).

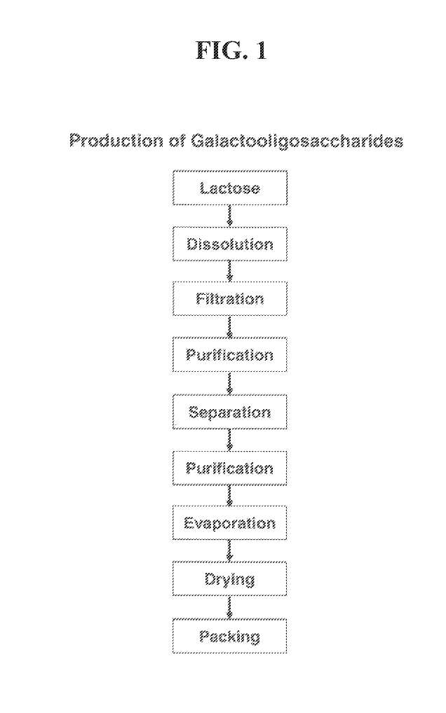

In some embodiments, the method may comprise additional purification steps. Purification techniques used may include filtration, de-colorization, evaporation, ion exchange and chromatographic separation (See FIG. 1). Other purification techniques such as centrifugation, membrane separation, crystallization, and electro-dialysis can also be used. In one embodiment, filtration is used to remove insoluble protein, which, if left in the final product, could solubilize and lead to off odors, poor taste, and rapid color (yellow) formation.

In some embodiments, the method further comprises the additional step of decolorizing the GOS solution with activated carbon. In some embodiments, the decolorization step is conducted prior to the three-column ion-exchange purification process. In some embodiments, the GOS syrup solution can be subjected to decolorization using a fixed-bed continuous decolorization system comprising an adsorption column packed with active carbon. Activated carbon is used to remove taste, color bodies, and odor. In some embodiments, the decolorization method comprises a fixed-bed continuous decolorization system using activated carbon. The organic impurities are adsorbed by the active carbon granules, which can be discharged, replaced by a fresh carbon layer, and regenerated in the furnace for later use.

As mentioned above, the present methods comprise the step of chromatographically purifying the galactooligosaccharide using an ion exchange resin comprising a K⁺ counter ion. In some embodiments, the ion exchange resin comprising a K⁺ counter ion is an acidic cation exchange resin with cross-linked polystyrene matrix and sulfonate functional groups. Representative resins which can be used with this chromatographic separation process include DIAION UBK-532 (MCI) or DOWEX Monosphere 99K 320 (Dow Chemicals).

The methods may include additional purification steps prior or after the chromatographic purification step. Thus, in some embodiments, the method further comprises single-column or multi-column ion-exchange purification process. In some embodiments, the ion-exchange purification process is conducted after the conversion of lactose to galactooligosaccharide and before the chromatographic purification step. In some embodiments, the process comprises the step of passing the GOS solution through at least one column selected from a cation column with strongly acidic cation exchange resin, an anion column with a basic anion exchange resin, and a mixed bed column comprising a combination of both strongly acidic and strongly basic resins. In some embodiments, the basic anion exchange resin is selected from a weakly basic anion exchange resin and an intermediate basic anion exchange resin. Thus, in some embodiments, the basic anion exchange resin is a weakly basic anion exchange resin. In other embodiments, the basic anion exchange resin an intermediate basic anion exchange resin.

In some embodiments, the method may further comprise a three-column ion-exchange purification process. In some embodiments, the process comprises passing the GOS solution through three ion-exchange columns. In some embodiments, the three-column ion-exchange purification process is conducted after the conversion of lactose to galactooligosaccharide. In some such embodiments, the three-column ion-exchange process is conducted prior to the chromatographic purification step. The ion-exchange columns can be cationic, anionic or mixed bed columns. In some embodiments, the three-columns may comprise a cation column with strongly acidic cation exchange resin, an anion column with a basic anion exchange resin; and a mixed bed column comprising a combination of both strongly acidic and strongly basic resins. In some embodiments, the basic anion exchange resin is selected from a weakly basic anion exchange resin and an intermediate basic anion exchange resin. Thus, in some embodiments, the basic anion exchange resin is a weakly basic anion exchange resin. In other embodiments, the basic anion exchange resin an intermediate basic anion exchange resin. These purification steps remove any ionic impurities, if present, e.g., calcium, chlorides, sulfates, phosphates, and other ionic components including amino acids, peptides and proteins from the GOS solution.

The strongly acidic cation-exchange resins, which are particularly useful in the practice of this technology, are those which contain sulfonate functional groups. These resins can conveniently be obtained by the copolymerization of compounds such as acrylic acid, methacrylic acid, acrylic esters, methacrylic esters, acrylonitrile or methacrylonitrile, and other unsaturated acrylates or nitriles with appropriate cross-linking agents such as divinylbenzene. In some embodiments, the resins have a cross-linked polystyrene matrix. Further illustrations of such resins useful in the practice of the present technology and of methods for their preparation may be found by reference to European Patent No. 0272095 and U.S. Pat. No. 5,130,239, which are incorporated herein by reference. Representative strong-acid cation exchange resins is Trilite AMP-24 (SamYang Corporation in Korea). In illustrative embodiments, the cation column comprises a strongly acidic cation exchange resin with a cross-linked polystyrene matrix, sulfonate functional groups and K⁺ or Na⁺ counter-ion such as, e.g., potassium polystyrene sulfonate or sodium polystyrene sulfonate.

The weakly-basic anion-exchange resins are generally resins having primary amine, secondary amine or tertiary amine as the principal functional group. A weakly basic anion exchange resin may be defined as one which has a pK in water falling in the range of 3.0-7.0. Typically the weak base polyamines are copolymers of acrylonitrile and methyl acrylate cross-linked with divinylbenzene and then subjected to aminolysis with polyamines; copolymers of styrene-divinylbenzene chloromethylates treated with primary or secondary amines; and reaction products of phenol-formaldehyde with a polyalkyleneamine. In some embodiments, the resins have a cross-linked polystyrene matrix. Further illustrations of such resins useful in the practice of the present technology and of methods for their preparation may be found by reference to U.S. Pat. No. 5,130,239, which is incorporated herein by reference. Representative weak-acid anion exchange resin is Trilite AW-90 (SamYang Corporation in Korea). In illustrative embodiments, the anion column comprises a weakly basic anion exchange resin with cross-linked polystyrene matrix, dimethylammonium functional groups and OH⁻ counter ion.

The intermediate-basic anion-exchange resins are generally resins comprising a mixture of primarily tertiary amine groups with a minor portion of quaternary amine groups. For example, an intermediate base anion exchanger may contain weak base anion exchangers and about 10-20% strong anion groups. These resins commonly have an aliphatic or polystyrene matrix. In some embodiments, the resins have a cross-linked polystyrene matrix. In illustrative embodiments, the anion column comprises an intermediate strength basic anion exchange resin with cross-linked polystyrene matrix and OH⁻ counter ion. The intermediate base anion exchange resins which can be utilized in the present technology are described in U.S. Pat. No. 4,015,939. Representative intermediate-acid anion exchange resins include, e.g., BIO-REX 5 (Bio-Rad Laboratories Inc.), Dowex XFS 40396 (Dow Chemical Company) and IONAC-A-305 (Ionac Chemical Company).

The mixed bed column used in the present technology generally involves a combination of strongly acidic cation exchange resins, such as those described above, and strongly-basic anion exchange resins. Examples of the strongly basic anion exchange resin which can generally be used in the present technology include resins obtained by chloromethylation of a styrene-divinylbenzene crosslinked copolymer, followed by amination of the product by dimethylethanolamine or trimethylamine and subsequently by forming a quaternary salt of the product of amination with halide as the counter ion. A representative strong-base anion exchange resin is DOWEX-22 manufactured by Dow Chemicals. In illustrative embodiments, the mixed bed column comprises a combination of strongly acidic cation exchange resin with cross-linked polystyrene matrix, sulfonate functional groups and K⁺ or Na⁺ counter-ion and strongly basic anion exchange resin with cross-linked polystyrene matrix, dimethylethanolammonium functional groups and Cl⁻ counter ion.

Following ion exchange purification, the GOS solution can be concentrated using an evaporator to produce a syrup. The concentrated GOS syrup can then be subjected to the chromatographic purification step using an ion exchange resin comprising a K⁺ counter ion as described above. The chromatographic column used for this step can be of a suitable diameter and length and comprises a strongly acidic cation exchange resin. The strongly acidic cation-exchange resin can be as described above but typically has a K⁺ counter ion. Representative strong-acid cation exchange resins having a K⁺ counter ion are DIAION UBK-532 [Mitsubishi Chemical Industry(MCI)] or DOWEX Monosphere 99K 320 (Dow Chemicals). This chromatographic purification step serves as a separation process where glucose, galactose, and lactose are separated from the GOS mixture. The separated products are recovered from the adsorbent bed through displacement with suitable solvents such as, e.g., sterilized/purified water. This chromatographic purification step may be repeated if required.

Although K⁺ counter ion is mentioned as the suitable counter ion in the chromatographic purification resin step, other forms of resin such as e.g., those having sodium, calcium magnesium, zinc or ammonium counter ions, or combinations thereof may also be readily used in the present methods.

In one embodiment, the present disclosure provides a method for producing high-purity galactooligosaccharide, the method comprising:

(a) contacting a lactose with a suitable enzyme under mildly acidic conditions to produce galactooligosaccharide;

(b) decolorizing galactooligosaccharide with activated carbon;

(c) passing galactooligosaccharide through one or more ion-exchange columns selected from a cation column with strongly acidic cation exchange resin, an anion column with a basic anion exchange resin, and a mixed bed column comprising a combination of both strongly acidic and strongly basic resins; and

(d) chromatographically purifying the galactooligosaccharide using an ion exchange resin comprising a K⁺ counter ion.

In various embodiments, the oligosaccharide product obtained by the present methods is composed of greater than about 85%, greater than about 90%, greater than about 92% of GOS, while the secondary fraction is composed of approximately about 3% to 7% lactose, about 10% to 15% oligosaccharide, about 20% to 25% galactose, and about 60% to 65% dextrose. The oligosaccharide fraction continues onto further processing, while the monosaccharide fraction is recycled back to glucose syrup. The oligosaccharide fraction can be further refined through a second round of ion exchange, activated carbon and evaporative concentration treatments. In an illustrative embodiment, the final purified and concentrated GOS product obtained comprises about 90% to about 92% oligosaccharide, about 5 to about 8% lactose, about 0% to about 3% dextrose, and about 0% to about 2% galactose. In some embodiments, the galactooligosaccharide composition may comprise no more than about 10% by weight of carbohydrates other than galactooligosaccharide.

In one aspect, a galactooligosaccharide composition obtained by the above methods is provided. In some embodiments, the galactooligosaccharide composition may comprise, as effective constituents, a mixture of one or more disaccharides, trisaccharides, tetrasaccharides, pentasaccharides and so on. In illustrative embodiments, the composition comprises from about 10% to about 25% w/v of the disaccharide, from about 30% to about 50% w/v of the trisaccharide, and from about 30% to about 45% w/v of the tetrasaccharide and higher oligosaccharides.

The product may then be rendered to a dry state in either crystalline or powdered form. In one embodiment, the product in a crystalline form may be produced through a series of unit operations of high density evaporation and crystallization utilizing chilling processes. In another embodiment, the product can be dried using a free-drying or evaporation technique, or spray dryers or belt dryers, resulting in a finished powder containing about 86% to about 98% GOS, and in some embodiments about 90% to about 98% GOS. In some embodiments, the finished product contains greater than about 90% GOS. In other embodiments, the finished product contains greater than about 92% GOS. In yet other embodiments, the finished product contains greater than about 95% GOS. The remaining composition typically comprises moisture and residual sugars. This product can be added to infant formula, food, feed and supplement preparations at low inclusion rates to show benefits. In some embodiments, the method is included in a process for producing a baby food, an infant formula, a beverage, a yogurt, or a dietary supplement.

Purity of the GOS composition may be evaluated using high performance liquid chromatography, sampling for taste testing, and spectrophotometry analysis for color and clarity.

Food Products Made with High Purity GOS Compositions and Methods for Producing Them

In another aspect, the present technology relates to food products made with high purity GOS, and methods for producing such food products. Thus, in one embodiment, a food product is provided comprising a galactooligosaccharide composition is provided. In some embodiments, the food product comprises: (a) a galactooligosaccharide composition comprising no more than about 10% by weight carbohydrates other than galactooligosaccharide; and (b) at least one additional edible ingredient. In some embodiments, the food products may also include a bulking agent. In some embodiments, the GOS compositions are characterized in that they include no more than about 15% by weight of carbohydrates other than GOS, no more than about 10% by weight of carbohydrates other than GOS, no more than about 8% by weight of carbohydrates other than GOS, and no more than about 5% by weight of carbohydrates other than GOS. As such, the present food products have better properties due to the lower inclusion level required to obtain an effective amount of GOS in the final food product. In some embodiments, the composition further comprises one or more oligosaccharides selected from the group consisting of fructooligosaccharides, isomaltooligosaccharides, and inulin.

The present food products include an oligosaccharide composition and at least one additional edible ingredient. Exemplary edible ingredients include proteins or protein hydrolysates, vegetable oils, other carbohydrates, pharmaceutically acceptable carriers, and other food additives such as those described below. In some embodiments, the food product may be in solid or liquid form. In some embodiments, the food product may take the form of a dry powder and include at least one salt or acid.

Food Additives. In addition to GOS, the food products provided herein will include a variety of additional synthetic and natural additives and components. The particular additives and components used will depend on the nature of the desired end product. However, by way of illustration, examples of the types of ingredients that may be included in the various food products described herein are provided below.

Saccharides, Oligosaccharides and Other Carbohydrates. In some embodiments, GOS can be used alone or in combination with other saccharides, oligosaccharides, or carbohydrates. Examples of further oligosaccharides include short-chain fructooligosaccharides, isomaltooligosaccharides, maltodextrin, and inulin. The food products of the present technology may further comprise another carbohydrate component. Examples of carbohydrate components include, but are not limited to, sucrose, high-fructose corn syrup, dextrose, hydrolyzed starch, polymerized glucose, maltose, glucose, lactose, fructose or combinations thereof.

Taste-Improving Compositions. A taste-improving composition is a compound or mixture that produces a food product having a more sugar-like taste or a sugar-like temporal profile than would be experienced if the taste-improving composition were not included in the food product. The taste-improving compositions include, but are not limited to, polyols, amino acids and their corresponding salts, polyamino acids and their corresponding salts, sugar acids and their corresponding salts, organic acids, inorganic acids, organic salts, inorganic salts, bitter compounds, flavorants, astringent compounds, polymers, proteins or protein hydrolysates, surfactants, emulsifiers, flavonoids, alcohols, synthetic sweeteners, and combinations thereof.

Natural High-Intensity Sweeteners. The food products of the present technology may comprise natural high-intensity sweeteners. Examples of natural high-intensity sweeteners include, but are not limited to, rebaudioside A, rebaudioside B, rebaudioside C, rebaudioside D, rebaudioside E, rebaudioside F, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, and combinations thereof.

Synthetic High-Intensity Sweeteners. The food products of the present technology may further comprise a synthetic high-intensity sweetener. Examples of synthetic high-intensity sweeteners include, but are not limited to, sucralose, acesulfame potassium and other salts, aspartame, alitame, saccharin, neohesperidin dihydrochalcone, cyclamate, neotame, N—[N-[3-(3-hydroxy-4-methoxyphenyl)propyl]-L-α-aspartyl]-L-phenylalanine 1-methyl ester, N—[N-[3-(3-hydroxy-4-methoxyphenyl)-3-methylbutyl]-L-α-aspartyl]-L-phenylalanine 1-methyl ester, N—[N-[3-(3-methoxy-4-hydroxyphenyl)propyl]-L-α-aspartyl]-L-phenylal-anine 1-methyl ester, salts thereof, and combinations thereof. However, some embodiments of the present food products will be free of synthetic sweeteners.

Antioxidants. The food products of the present technology may further comprise antioxidants. Types of antioxidants include ascorbic, isoascorbic, eritorbic, and citric acid types. Other examples include sodium, calcium, and potassium ascorbate; soybean lecithin; esters of citric acid and fatty acids with glycerol; esters of citric and mono- and di-glyercerides; enzymes such as glucose-oxidase (Aspergillus niger); ascorbil palmitate, ascorbil stearate; a concentrated mix of tocopherols or tocopherols and alpha-tocopherol; propyl galate; tert-butyl hydroquinone (TBHQ); butyl hydroxyanisol (BHA); butyl hydroxytoluene (BHT); isopropyl citrate (mix); and isopropyl citrate (mono).

Conservants. The food compositions of the present technology may further comprise a conservant. Examples of conservants include, but are not limited to, propionic and acetic acid; sodium, calcium, and/or potassium propionate; sodium erithorbate; isoascorbate; and calcium acetate.

Salts. The food compositions of the present technology may further comprise a salt. Examples of salts include, but are not limited to, inorganic magnesium salts (sodium, potassium, calcium, or magnesium salt), inorganic phosphate salts (sodium, potassium, calcium, or magnesium phosphate), and inorganic chloride salts (sodium, potassium, calcium, or magnesium chloride).

Organic and Inorganic Acids. The food compositions of the present technology may further comprise an organic and/or inorganic acid. Examples of these acids include, but are not limited to, tartaric, adipic, phosphoric, lactic, citric, ascorbic, gluconic malic, fumaric, or tartaric acid, or combinations thereof.

Emulsifiers and Stabilizers. The food compositions of the present technology may further comprise an emulsifier/stabilizer. Examples of emulsifiers and stabilizers include, but are not limited to, propylene glycol alginate, polyethylene stearate, sorbitan derivatives (polyoxyethylene stearate, polyoxyethylene monooleate, polyoxyethylene monolaurate, polyoxyethylene monopalmitate, polyoxyethylene monostearate, polyoxyethylene tristearate, stearate, monooleate, tristearate, monopalmitate), sodium stearoyl-2-lactylate, calcium stearoyl-2-lactylate, fatty acid esters with propylene glycol, tartaric diacetyl acid esters and fatty acids with glycerol, tartaric diacetyl acid esters and mono and diglycerides, lecithin, sodium caseinate, citrate (sodium, monosodium, disodium and trisodium), gums (xanthan, guar, adragante, arabic, konjac), mono and diglycerides, sorbitol, cellulose derivates (methyl, methyl ethyl, hydroxypropyl, microcrystalline), sodium carboxymethyl cellulose, and salts of fatty acids (calcium, sodium, potassium, ammonium).

Polyols. The food compositions of the present technology may further comprise a polyol. Examples of polyols include, but are not limited to, erythritol, xylitol, sorbitol, maltitol, lactitol, mannitol, isomalt, polydextrose, and hydrogenated starch hydrolysates or combinations thereof.

Flavor Enhancers. The food products of the present technology may further comprise a flavor enhancer. Examples of flavor enhancers include, but are not limited to, glutamic acid and its salts, guanilic acid and its salts, inosinic acid and its salts, or combinations thereof.

Protein, Protein Hydrolysates, Amino Acids. The food compositions of the present technology may further comprise a protein or amino acid component. Examples of protein or amino acid components include, but are not limited to, whey protein isolates, soy protein isolates, whey protein concentrates, soy protein concentrates, and their hydrolysates; or an amino acid or its corresponding salts (glycine, alanine, proline, hydroxyproline, glutamine, or combinations thereof), polyamino acids (poly-L-aspartic acid, poly-L-α-lysine, poly-L-ε-lysine, poly-L-α-ornithine, poly-ε-ornithine, poly-L-arginine, salts thereof), or combinations thereof.

Bulking agents. The term “bulking agents,” as used herein, may be any of those typically used in the art and include polydextrose, cellulose and its derivatives, maltodextrin, corn syrup solids, sucrose, fructose, glucose, invert sugar, sorbitol, xylose, ribulose, mannose, xylitol, mannitol, galactitol, erythritol, maltitol, lactitol, isomalt, maltose, tagatose, lactose, inulin, glycerol, propylene glycol, polyols, polydextrose, pectin, alginate, gum arabic, xantham, guar, gelan, carragenan, gelatin, starch, modified starch, and the like, or combinations thereof.

Milk Solids. The food products of the present technology may further comprise a milk solid. Examples of milk solids include, but are not limited to, whole milk powder, milk fat, or skim milk, and include whey powders and whey concentrates or combinations thereof.

Minerals and Vitamins. The food products of the present technology may further comprise minerals and vitamins, including, but not limited to, calcium, iron, selenium, zinc, ascorbic acid, β-carotene, and others.

Inorganic Salts. The food products of the technology may further comprise inorganic salts. The inorganic salts may comprise sodium, potassium, calcium, magnesium, or phosphate salts.

Non-limiting examples of food products that may be made with the high purity GOS compositions include infant formula, baby and toddler foods, beverages, yogurts, and nutritional supplements. In some embodiments, the food product comprising galactooligosaccharide is a beverage and the additional edible ingredient comprises at least one protein or protein hydrolysate, vegetable oil, and at least one additional carbohydrate. In some embodiments, the food product comprising galactooligosaccharide is yoghurt and the additional edible ingredients comprise at least one protein or protein hydrolysate, milk, at least one acid, and at least one additional carbohydrate. In some embodiments, the food product comprising galactooligosaccharide is a baby food and the additional edible ingredients comprise at least one protein or protein hydrolysate and at least one additional carbohydrate. In some embodiments, the food product comprising galactooligosaccharide is an infant formula and the additional edible ingredients comprise at least one protein or protein hydrolysate, vegetable oil, and at least one additional carbohydrate. In some embodiments, the food product comprising galactooligosaccharide is a dietary supplement and the additional edible ingredients comprise a pharmaceutically acceptable carrier.

In another aspect, a method of preparing a food product comprising a galactooligosaccharide composition is provided. In some embodiments, the method comprises mixing a galactooligosaccharide composition comprising no more than about 10% by weight of carbohydrates other than galactooligosaccharide and at least one additional edible ingredient. In some embodiments of the method, the GOS compositions are characterized in that they include no more than about 15% by weight of carbohydrates other than GOS, no more than about 10% by weight of carbohydrates other than GOS, no more than about 8% by weight of carbohydrates other than GOS, and no more than about 5% by weight of carbohydrates other than GOS. In some embodiments, the composition further comprises one or more oligosaccharides selected from the group consisting of: fructooligosaccharides, isomaltooligosaccharides, and inulin. The edible ingredients used in the present methods are as described above. The method can be used to produce various food and nutritional products including infant formula, baby and toddler foods, beverages, yogurts, and nutritional supplements.

Food products that may benefit from the inclusion of the present GOS compositions and that may be produced using the present methods include any food products provided to individuals in need of the nutritional benefits (e.g., improved gastrointestinal health) that GOS provide. Food products may also benefit from the inclusion of GOS compositions in other ways, including, for example, flavor enhancement, moisture retention, and extended shelf-life. GOS are highly stable ingredients that withstand heat processing conditions such as retort, pasteurization, and UHT (ultra-high temperature) pasteurization. Moreover, GOS are stable over a range of pH and can be added to both neutral and low pH food and beverages. The stability of GOS allows for inclusion at the initial stages of processing.

Examples of such food products include milk beverages (powdered or ready to drink), soy beverages (powdered or ready to drink), functional beverages, yogurt, baby foods, baby and toddler foods, infant formula (prepared or ready to feed), or nutritional supplements. These products are described generally below and in Table 1, and in greater detail in the Examples section which follows.

TABLE 1
Exemplary Uses Levels of High Purity GOS

| | | Serving | GOS Levels | |
|---|---|---|---|---|
| | Serving | Size* | (g per | Inclusion |
| Application | Size* | (g) | serving) | Level (%) |
| Milk Beverages | 8 fl oz | 237 | 1.3-1.5 | 0.6 |
| Soy Beverages | 8 fl oz | 237 | 1.3-1.5 | 0.6 |
| Functional Beverages | 8 fl oz | 237 | 1.3-1.5 | 0.6 |
| Yogurt | 1 cup (8 oz) | 225 | 1.3-1.5 | 0.6-0.7 |
| Baby Foods | 2 oz | 56.7 | 1.4 | 2.5 |
| Baby and Toddler | 4 oz | 113 | 1.3-1.5 | 1.2-1.3 |
| Foods | | | | |
| Supplements, capsules | 2 capsules | 1.5 | 1.3-1.5 | 87.6-99.8 |
| Infant Formula | 5 fl oz, | 148 | 0.6 | 0.4 |

*Serving sizes based on Reference Amounts Customarily Consumed per Eating Occasion (RACC; 21 CFR § 101 .I2 - U.S. FDA, 2008).

The effective amount of high-purity GOS composition in a given food product or dietary supplement will vary depending upon the nature, desired flavor and desired consistency of the food product. The term “effective amount” is meant a quantity sufficient to achieve a desired nutritional, therapeutic and/or prophylactic effect, e.g., an amount which results in the prevention of, or a decrease in, the symptoms associated with a condition, e.g., a deficiency in certain gastrointestinal flora such as Bifidobacteria. The amount of a composition of the technology administered to the subject will depend on the type and severity of the condition and on the characteristics of the individual, such as general health, age, sex, body weight, etc. The skilled artisan will be able to determine appropriate dosages depending on these and other factors. The amount will also depend on the nutritional effect desired.

The GOS compositions provide the ability to include a relatively low amount of the composition in a food product (e.g., no greater than 0.1 percent, no greater than 1 percent, no greater than 5 percent, or no greater than 10 percent based on the dry weight of the ingredients) so as not to interfere with the properties of the other ingredients in the food product. Alternatively, the present GOS compositions may be added in a relatively high amount (e.g., at least 1 percent, at least 5 percent, at least 10 percent, or at least 25 percent, based on the dry weight of the ingredients), in order to provide food products with an enhanced nutritional benefit.

The GOS composition as a food ingredient can be used in a variety of food products including baby, infant and toddler foods, beverages and beverage bases, dairy product analogs, milk products, bakery products, beverages, cereal and other grain products, desserts, fruit and fruit juices, snacks, soups, and soft and hard candy, at use levels of 0.48% to 12.21% per serving. Under infant formula comprising GOS, wherein GOS is intended for use as a food ingredient in infant formula and follow-on formula, GOS is usually present at concentrations not exceeding 0.72% (7.2 g of GOS per liter of infant formula) of the final reconstituted or ready-to-serve product.

Infant Formula. Infant formulas are nutritional compositions designed for children 1 year, or younger, which contains sufficient protein, carbohydrate, fat, vitamins, minerals, and electrolytes to serve as the sole source of the nutrition for these children, when provided in a sufficient quantity. Infant formula is typically available as a ready-to-feed liquid, a concentrate that is diluted prior to consumption, or a powder that is reconstituted prior to consumption.

GOS is naturally occurring in human milk. Thus, infant formulas can be developed using GOS to closer match the nutritional profile of breast milk. High purity GOS can be used alone or in combination with other oligosaccharides such as, short-chain fructooligosaccharides, isomaltooligosaccharides and inulin. Typical ingredients in infant formula compositions may include whey protein isolates, whey protein concentrates, whey protein hydrolysates, soy protein isolates, soy protein concentrates, soy protein hydrolysates, vegetable oils, such as coconut oil, palm oil, soybean oil, safflower oil, and sunflower oil, lactose, sucrose (used in lactose free products), maltodextrin, cornstarch, vitamin and mineral blends, nucleotides, probiotic cultures, omega-3 fatty acids, soy lecithin (emulsifier), and carrageenan (stabilizer used in ready-to-feed preparations).

Milk, Soy Drinks, and Functional Beverages. Ready-to-drink beverages are typically provided in a shelf-stable dry form, which may be combined with water or another liquid immediately prior to consumption. Alternatively, the dry form may be combined with water by the manufacturer and packaged for sale to the consumer as a ready-to-drink beverage. Such beverage mixes and beverages are well-known and come in a variety of flavors and colors. Included in this category are “functional beverages” which are beverages fortified with dietary supplements (e.g., GOS) and herbal medicines. Beverages can be prepared with high purity GOS to deliver prebiotic and immune enhancing benefits.

Dairy Compositions. High-purity GOS compositions may be added to dairy compositions generally. Dairy compositions comprise dairy desserts, milk, or foodstuffs produced from milk, such as cream, sour cream, buttermilk, cultured buttermilk, milk power, condensed milk, sweetened condensed milk, evaporated milk, butter, cheese, cottage cheese, cream cheese, and yogurt. Examples of dairy desserts include, but are not limited to, ice cream, frozen custard, frozen yogurt, gelato, and ice milk. The compositions may further include amino acids, organic and/or inorganic salts, carbohydrates, synthetic sweeteners, and bulking agents.

Baby and Toddler Foods. Dry and high moisture baby and toddler foods may be prepared with high purity GOS. These may include baked snacks, puffed snacks, extruded cereals and the like, ready-to-feed vegetable and fruit purees, combination dishes, and prepared meals.

Yogurt. Yogurts can be formulated with high purity GOS to deliver the benefits of probiotic cultures along with prebiotic fiber. Such products are made from a fermentation mixture obtained from milk or soymilk using live cultures. These products may take on a variety of solid, semi-solid, and liquid forms. Yogurts are typically made with at least one of the following microorganisms: Lactobacillus acidophilus, Bifidobacterium lactis, Streptococcus thermophilus, Lactobacillus bulgaricus, Lactobacillus casei, Bifidobacterium bifidum, Lactobacillus bifidus, Lactobacillus lactis, or combinations thereof.

Dietary Supplements. High purity GOS can be added at low inclusion rates to deliver health benefits in a supplement form, such as tablets, capsules and sachets. In addition, the supplement composition may contain pharmaceutically acceptable carriers, fillers, coatings, binders, disintegrates, lubricants, processing aids and the like, such as stearates, silicates, maltodextrin, starches, steraric acid, cellulose, gelatin, flavors, colors and sweeteners. The GOS may be administered in a powdered, reconstitutable powder, liquid-solid suspension, liquid, capsule, tablet, and caplet dosage forms. It should be readily obvious to one of ordinary skill in the science of formulations that the present dietary supplement can also be formulated appropriately for various forms of administration. Thus, other dosage forms such as chewable candy bar, concentrate, drops, elixir, emulsion, film, gel, granule, chewing gum, jelly, oil, paste, pastille, pellet, shampoo, rinse, suppository, syrup, chewable gelatin form, or chewable tablet can be used. In one embodiment, the GOS is formulated in a capsule.

Due to varying diets among people, the dietary supplement of the technology can be administered in a wide range of dosages and formulated in a wide range of dosage unit strengths. For those people who may be in need of improved gastrointestinal health, a dietary supplement containing GOS in nutritionally effective amounts can be formulated.

It should be noted that the dosage of the dietary supplement can vary according to a particular condition of the subject. In one embodiment, the recommended daily intake for GOS for adults would be from about 2 g to about 4 g, or from about 2 g to about 3 g, in order to deliver benefits for immune health. The recommended daily intake of GOS for infants would be from about 2 g to about 4 g, or from about 3 g to about 4 g, in order to deliver benefits for immune health. An appropriate dose of the dietary supplement can be readily determined by monitoring subject response, i.e., general health, to particular doses of the supplement. As well, another agent such as a vitamin, mineral, nutrient, phytonutrient, plant extract, or herbal extract may be administered to a subject along with the present GOS dietary supplement. In such cases, the appropriate doses of the supplement and each of the agents can be readily determined in a like fashion by monitoring subject response, i.e., general health, to particular doses of each.

It is contemplated by the technology that the dietary supplement can be administered simultaneously or sequentially in one or a combination of dosage forms. While it is possible that the present dietary supplement will provide an immediate overall health benefit, such benefit may take days, weeks or months to materialize. Nonetheless, the present GOS dietary supplement may provide a beneficial nutritional response in a subject consuming it.

The dietary supplement may also be combined with one or more pharmaceutically acceptable carriers. As used herein, the term “pharmaceutically acceptable carrier” is intended to include any and all solvents, dispersion media, coatings, antibacterial and antifungal compounds, isotonic and absorption delaying compounds, and the like, compatible with pharmaceutical administration.

### EXAMPLES

The present methods, thus generally described, will be understood more readily by reference to the following examples, which are provided by way of illustration and are not intended to be limiting of the present methods. The following is a description of the materials and experimental procedures used in the Examples.

### Example 1

### High Purity GOS Preparations

Lactose powder was dissolved in heated water 80-85° C. in a saccharification tank. When the concentration of lactose on a dry solid (DS) basis was 40-55%, the temperature of the solution was reduced to 60-65° C., and the pH is adjusted to mildly acid conditions (pH between 5.0 and 6.5) using sodium hydroxide (NaOH) and hydrochloric acid (HCl) solutions as required. The B. circulans LOB 377 enzyme was then added to the solution where it reacted with lactose to produce GOS. Saccharification was continued in the stirred solution tank over a 2-day period until the desired oligosaccharide content (more than 37% w/v) was achieved. The hydrolysate formed during saccharification was then pumped through a heat exchanger where the solution was heated to 85-90° C., resulting in inactivation of the β-galactosidase enzyme. The inactivated enzyme was removed via a Celite filter (plankton diatomite), and the product was decolorized using a fixed-bed continuous decolorization system. The organic impurities were adsorbed by the active carbon granules, which were discharged, replaced by a fresh carbon layer, and regenerated in the furnace for later use. The decolorized solution was then cooled via a heat exchanger and then proceeds through a three-column ion exchange purification process. The solution was first passed through a cation column with strongly acidic cation exchange resin; followed by a anion column with a weakly basic or an intermediate basic anion exchange resin; and finally a mixed bed column that has a combination of both strongly acidic and strongly basic resins, thereby removing any ionic impurities. Following active carbon and ion exchange purification, the GOS solution was concentrated using an evaporator to produce a syrup (approximately 50% to 60% w/v of saccharides). The concentrated GOS syrup was then subjected to a chromatographic separation process where glucose, galactose, and lactose were separated from the GOS mixture. This was done by passing the concentrated GOS syrup through a chromatography column of strongly acidic cation exchange resin comprising a cross-linked polystyrene matrix, sulfonate functional group and K⁺ counter ion. The separated products were recovered from the adsorbent bed through elution with sterilized purified water. Following chromatographic purification, the oligosaccharide fraction is composed of greater than 90% GOS, while the secondary fraction is composed of approximately 3% to 7% lactose, 10% to 15% oligosaccharide, 20% to 25% galactose, and 60% to 65% dextrose. The oligosaccharide fraction was subjected to further processing, while the monosaccharide fraction was recycled back to glucose syrup. The oligosaccharide fraction was further refined through a second round of ion exchange, activated carbon and evaporative concentration treatments. The product was obtained in greater than 40% yield. The composition of the GOS product was analyzed using high-performance liquid chromatography. The final purified and concentrated composition of the GOS syrup had approximately 90% to 92% oligosaccharide, 5% to 8% lactose, 0% to 3% dextrose, and 0% to 2% galactose. The syrup was then heated by passing through a heat exchanger and subjected to drying using techniques such as evaporation, freeze drying or hot air spray drying, whereby the final product, a purified white GOS powder (97% dry solids) was obtained. The final product was packaged in poly-lined craft paper bags and stored at room temperature. The final product had the following specifications:

TABLE 2
GOS (90-92% Purity)

| | | Content (% | |
|---|---|---|---|
| Carbohydrates | | dry basis) | Structure |
| Monosaccharides | Fructose | 0-1.0 | D-fru |
| | Glucose | 0-3.0 | α-D-glc |
| | Galactose | 0-2.0 | β-D-gal |
| Disaccharides | Lactose | 5.0-8.0 | β-D-gal-(1,4)-D-glc |
| | GOS | 2.0-5.0 | β-D-gal-(1,3)-D-glc; |
| | | | β-D-gal-(1,4)-D-gal |
| | GOS | 5.0-10.0 | β-D-gal-(1,6)-D-glc |
| Trisaccharides | GOS | 15.0-20.0 | β-D-gal-(1,3)-β-D-gal- |
| | | | (1,4)-β-D-glc |
| | GOS | 15.0-20.0 | β-D-gal-(1,4)-β-D-gal- |
| | | | (1,4)-β-D-glc |
| | GOS | 6.0-8.0 | β-D-gal-(1,6)-β-D-gal- |
| | | | (1,4)-β-D-glc |
| Tetrasaccharides | GOS | 34.0-41.0 | β-D-gal-[(1,6)-β-D-gal- |
| & higher | | | (1,3)]n-β-D-gal-(1,4)-D-glc |
| oligomers n ≧ 3 | | | |

| | |
|---|---|
| Total GOS | 90.0-92.0 |

GOS = Galactooligosaccharide;
Gal = Galactose;
Glc = Glucose

In accordance with the present technology, high purity GOS compositions were prepared. FIG. 1 shows a schematic of the GOS preparation process. The final concentration of GOS and other saccharides in composition can be varied by varying the chromatographic purification parameters and by repeating the purification steps. Examples of two compositions produced by these methods are shown in Tables 3 and 4. The composition in Table 3 has a final GOS purity of approximately 90-93% by weight by weight and the composition in Table 4 has a final GOS purity of approximately 95% by weight.

TABLE 3
GOS (90-93% Purity)

| | Content | |
|---|---|---|
| Component | (% dry basis) | Structure |
| Dextrose | 0-1.0 | α-D-glc |
| Galactose | 0-0.5 | β-D-gal |
| Lactose | 7.0-10.0 | β-D-gal-(1,4)-D-glc |
| Galactooligosaccharides | 90.0-93.0 | |
| Glu-Gal-Gal (4′-Gal) | 16.0-20.0 | β-D-gal-(1,4)-β-D-gal-(1,4)-D- |
| | | glc |
| Glu-Gal-Gal (6′-Gal) | 8.0-13.0 | β-D-gal-(1,6)-β-D-gal-(1,4)-D- |
| | | glc |
| Glu-Gal-Gal-Gal ≧ | 25.0-29.0 | β-D-gal-[(1,6)-β-D-gal-(1,3)]n- |
| | | β-D-gal-(1,4)-D-glc |

TABLE 4
GOS (95-97% Purity)

| | Content | |
|---|---|---|
| Component | (% dry basis) | Structure |
| Dextrose | 0-1.0 | α-D-glc |
| Galactose | 0-0.5 | β-D-gal |
| Lactose | 2.0-5.0 | β-D-gal-(1,4)-D-glc |
| Galactooligosaccharides | 95.0-97.0 | |
| Glu-Gal-Gal (4′-Gal) | 19.0-23.0 | β-D-gal-(1,4)-β-D-gal-(1,4)-D- |
| | | glc |
| Glu-Gal-Gal (6′-Gal) | 8.0-13.0 | β-D-gal-(1,6)-β-D-gal-(1,4)-D- |
| | | glc |
| Glu-Gal-Gal-Gal ≧ | 35.0-40.0 | β-D-gal-[(1,6)-β-D-gal-(1,3)]n- |
| | | β-D-gal-(1,4)-D-glc |

### Example 2

### Food Compositions Comprising High Purity GOS

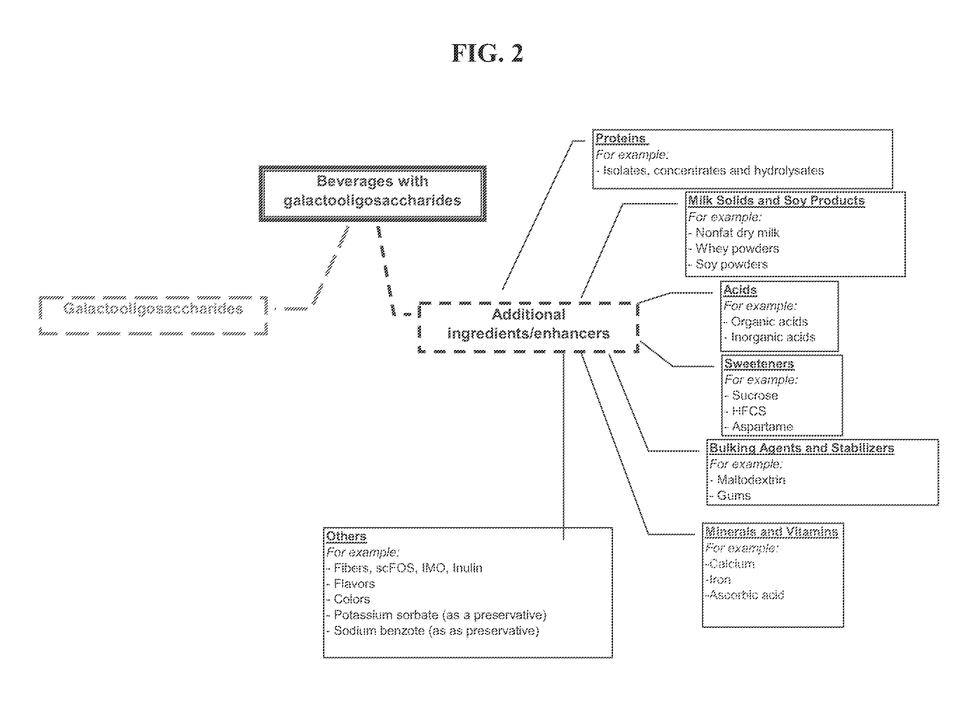

Beverages. The high-purity GOS composition of the present technology may be included in a beverage mix. The basic blend includes GOS with proteins, acids, sweeteners, bulking agents, and vitamins and minerals. Optionally acids, salts, flavorings, and coloring agents may be added. Variations on the basic blend may be made using one or more of the following ingredients: proteins and protein hydrolysates, salts, acids, carbohydrates, bulking agents, polyols, and flavor-masking agents (see FIG. 2). The ingredients may be mixed in their dry form. The product may be provided as a dry mix concentrate to be incorporated in a beverage formulation.

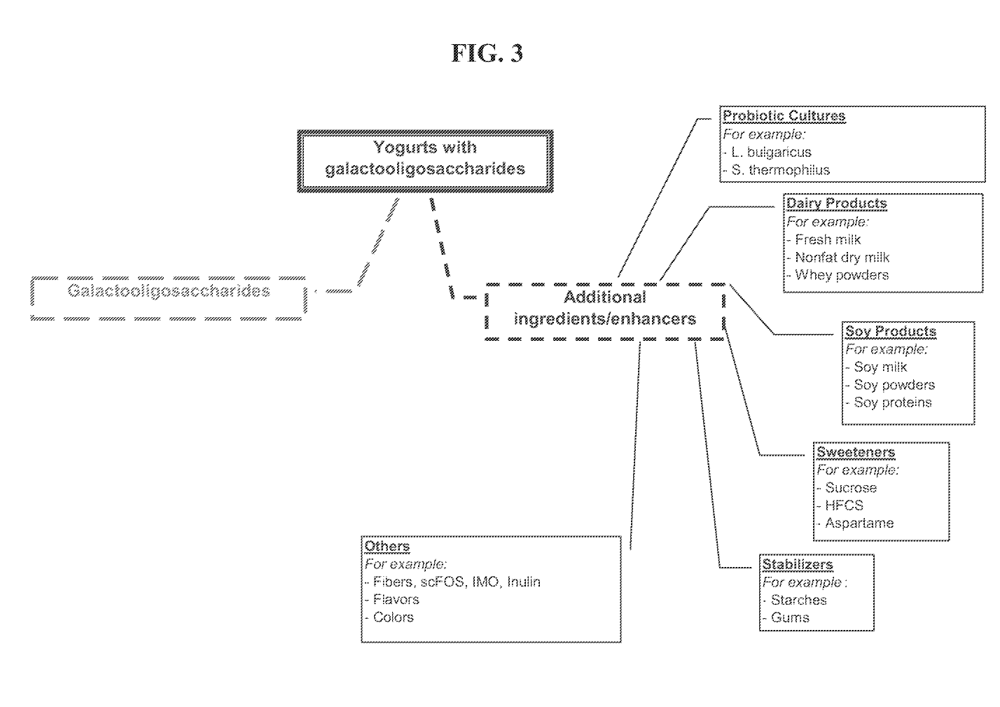

Yogurt. A yogurt food composition comprising a high-purity GOS composition may be made by fermenting a mixture obtained from milk or soymilk using live culture organisms. The yogurt beverage may contain flavor enhancers, colorings, thickeners, fruit preparations, and a high-purity GOS composition. Variations of the mixture may be made using a combination of the ingredients shown in FIG. 3.

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as “up to,” “at least,” “greater than,” “less than,” and the like, include the number recited and refer to ranges which can be subsequently broken down into subranges as discussed above. Finally, as will be understood by one skilled in the art, a range includes

All references cited herein are incorporated by reference in their entireties and for all purposes to the same extent as if each individual publication, patent, or patent application was specifically and individually incorporated by reference in its entirety for all purposes.

The technology illustratively described herein may suitably be practiced in the absence of any element or elements, limitation or limitations, not specifically disclosed herein. Thus, for example, the terms “comprising,” “including,” containing,” etc., shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the technology claimed.

Other embodiments are indicated by the following claims.

What is claimed is:

1. A method of producing galactooligosaccharide comprising catalyzing the conversion of lactose to galactooligosaccharide using a suitable enzyme; and chromatographically purifying the galactooligosaccharide using an ion exchange resin comprising a K⁺ counter ion.

2. The method of claim 1, wherein the enzyme is a β-galactosidase.

3. The method of claim 2, wherein the β-galactosidase is derived from Cryptococcus laurentii, Aspergillus oryzae, Aspergillus nigar; Bacillus circulans, Bacillus subtilis, Bacillus licheniformis, Lactobacillus bulgaricus, Streptococcus thermophilus, Bullera singularis, Bifidobacterium breve, Bifidobacterium longum, Bifidobacterium infantis, Bifidobacterium bifidum, Lactococcus lactis, Candida pseudotropicalis, or Kluyveromyces lactis.

4. The method of claim 3, wherein the β-galactosidase is derived from Bacillus circulans.

5. The method of claim 1, wherein the ion exchange resin comprising a K⁺ counter ion is an acidic cation exchange resin with cross-linked polystyrene matrix and sulfonate functional groups.

6. The method of claim 1, further comprising the step of passing the galactooligosaccharide through atleast one ion-exchange column selected from a cation column with strongly acidic cation exchange resin, an anion column with a basic anion exchange resin, and a mixed bed column comprising a combination of both strongly acidic and strongly basic resins.

7. The method of claim 6, wherein the basic anion exchange resin is selected from a weakly basic anion exchange resin or an intermediate basic anion exchange resin.

8. The method of claim 6, comprising a three-column ion-exchange purification process.

9. The method of claim 8, wherein the three-columns comprise a cation column with strongly acidic cation exchange resin, an anion column with a basic anion exchange resin; and a mixed bed column comprising a combination of both strongly acidic and strongly basic resins.

10. The method of claim 9, wherein the basic anion exchange resin is selected from a weakly basic anion exchange resin or an intermediate basic anion exchange resin.

11. The method of claim 8, wherein the three-column ion-exchange process is conducted prior to the chromatographic purification step.

12. The method of claim 1, further comprising the additional step of decolorizing the galactooligosaccharide solution with activated carbon prior to the ion-exchange purification step.

13. The method of claim 1, wherein an aqueous solution of lactose is contacted with the enzyme.

14. The method of claim 13, wherein the concentration of lactose in the solution ranges between about 5% and about 90%.

15. The method of claim 1, wherein the galactooligosaccharide composition comprises no more than about 10% by weight of carbohydrates other than galactooligosaccharide.

16. The method of claim 1, wherein the method is included in a process for producing a baby food, an infant formula, a beverage, a yogurt, or a dietary supplement.

17. A method for producing high-purity galactooligosaccharide, the method comprising:
(a) contacting lactose with a suitable enzyme under mildly acidic conditions to produce galactooligosaccharide;
(b) decolorizing galactooligosaccharide with activated carbon;
(c) passing galactooligosaccharide through one or more ion-exchange columns selected from a cation column with strongly acidic cation exchange resin, an anion column with a basic anion exchange resin, and a mixed bed column comprising a combination of both strongly acidic and strongly basic resins; and
(d) chromatographically purifying the galactooligosaccharide using an ion exchange resin comprising a K⁺ counter ion.

18. A galactooligosaccharide composition obtained by the process of claim 17.

19. The galactooligosaccharide composition of claim 18 comprising, as effective constituents, a mixture of one or more disaccharides, trisaccharides, tetrasaccharides and pentasaccharides.

20. The galactooligosaccharide composition according to claim 19, comprising from about 10% to about 25% w/v of the disaccharide, from about 30% to about 50% w/v of the trisaccharide, and from about 30% to about 45% w/v of the tetrasaccharide and higher oligosaccharides.
